(19) United States
(12) Patent Application Publication
(10) Pub. No.: US 2010/0030134 A1
(43) Pub. Date: Feb. 4, 2010

(54) PRECISION ORIFICE SAFETY DEVICE

(76) Inventors: Matthew J. Fitzgerald, St. Louis, MO (US); Toh Seng Goh, Ballwin, MO (US)

Correspondence Address: Bausch & Lomb Incorporated, One Bausch & Lomb Place, Rochester, NY, 14604-2701, US

(21) Appl. No.: 12/181,673

(22) Filed: Jul. 29, 2008

(51) Int. Cl. A61M 1/00

(52) U.S. Cl. 604/34; 604/30; 604/35

(57) ABSTRACT

A safety device in a fluid vent conduit for reducing vacuum level in an aspiration conduit of an ophthalmic surgical system. The device includes an orifice having an inlet for receiving a flow of fluid from an irrigation source. The orifice further has an outlet for directing the flow of fluid into the aspiration conduit. The orifice has a cross-sectional area sufficiently large to ensure an acceptable amount of fluid flows into the aspiration conduit, to reduce the vacuum level in the aspiration conduit, to an acceptable level in an acceptable amount of time and to provide adequate reflux. The cross-section area is sufficiently small to restrict the flow of fluid to allow enough fluid to continue to flow from the irrigation source to a handpiece at a surgical site.

### FIELD

The present invention relates generally to a device for controlling a fluid flow in a system used in various surgical procedures. More specifically, it relates to a safety device for providing improved fluid control in an ophthalmic surgical system to maintain an appropriate intra-pressure within a conduit of the surgical system.

### BACKGROUND

A cataract is an opacity that develops in the crystalline lens of the eye or in its envelope. One medical procedure to remove a cataract-affected lens is phacoemulsification using ultrasonic sound to break up or emulsify the cataract. A phacoemulsification machine typically includes a handpiece with both irrigation and aspiration functions. A phaco handpiece sucks-in or aspirates emulsified fluids and simultaneously replaces those aspirated fluids with irrigation fluid, typically balanced salt solution (BSS), to maintain a proper pressure of the anterior chamber of the patient's eye. Such a handpiece is connected to a pump generating negative pressure or vacuum to drive aspiration, by which debris from the eye flow through a tube to means for collection such as a cassette, a bag, or a bottle.

A common and dangerous occurrence in ophthalmic surgery is post-occlusion surge. During ophthalmic surgery, particularly cataract surgery, as the lens is broken-up and emulsified, such as during phacoemulsification, irrigation fluid is constantly infused into the surgical site and the fluid and emulsified tissue are aspirated away from the surgical site through the phaco handpiece. On occasion bits of tissue are larger than the aspiration lumen at the tip of the phaco handpiece, which can occlude the tip and cause an increase in vacuum in the aspiration conduit. As long as the tip remains occluded, a negative pressure builds up throughout the aspiration system. Then, after the occlusion has been removed, the system can experience what is commonly referred to as surge. Post-occlusion surge can cause serious damage to a patient's eye.

One way to reduce such risks is to provide a bypass circuit linking an irrigation line and an aspiration line of an ophthalmic surgical system so that a negative pressure built inside the aspiration line can be neutralized by irrigation fluid supplied from an irrigation source. This is commonly referred to as fluid venting. To achieve the goal effectively with the approach, however, it is required to control the influx amount of the irrigation fluid so as to flow just enough to equalize the negative pressure. Generally speaking, uncontrolled-bypassing flow could result in undesirable effects in removing cataract. For example, under-influx could not be effective to equalize the negative pressure built therein and over-influx could prevent sufficient amount of the irrigation fluid from flowing to a handpiece. Therefore, there is a need to eliminate or reduce such undesirable effects arising from uncontrolled influx of an irrigation fluid.

Venting, whether fluid or air, is typically done to the aspiration circuit whenever aspiration is intentionally halted during surgery. An example of this is when a surgeon moves from foot pedal position 2 or 3 back to position 1. This venting is done to remove any residual vacuum in the aspiration circuit and return to atmospheric pressure.

### SUMMARY OF THE INVENTION

It is therefore one of the objects of this invention to provide a safety device in a fluid vent path for an ophthalmic surgical system to provide improved fluid flow control.

In one embodiment, it is provided a safety device in a fluid vent conduit for reducing vacuum level in an aspiration conduit of an ophthalmic surgical system comprising an orifice having an inlet for receiving a flow of fluid from an irrigation source, wherein the orifice further has an outlet for directing the flow of fluid into the aspiration conduit, and wherein the orifice has a cross-sectional area sufficiently large to ensure an acceptable amount of fluid flows into the aspiration conduit, to reduce the vacuum level in the aspiration conduit, to an acceptable level in an acceptable amount of time and to provide adequate reflux, and wherein the cross-section area is sufficiently small to restrict the flow of fluid to allow enough fluid to continue to flow from the irrigation source to a handpiece at a surgical site.

In another embodiment, it is provided an ophthalmic surgical pump cassette for collecting aspirant fluid and tissue from a patient's eye which comprises an aspiration manifold formed on the cassette for connecting an aspiration conduit from a surgical handpiece to the cassette and for allowing valves of a surgical console to control a flow of aspirant fluid and tissue into the cassette; an irrigation manifold formed on the cassette for connecting an irrigation conduit from a surgical handpiece to a source of irrigation fluid and for connecting a fluid vent conduit between the aspiration manifold and the irrigation manifold, the irrigation manifold also allowing valves of the surgical console to control a flow of irrigation fluid from the source to the handpiece and between the irrigation manifold and the aspiration manifold; and an orifice formed within the fluid vent conduit or a manifold connecting the fluid vent conduit and the aspiration conduit, the orifice having a cross-sectional area sufficiently large to ensure an acceptable amount of fluid flows into the aspiration manifold to reduce a vacuum level in the aspiration conduit, to an acceptable level in an acceptable amount of time, and to provide adequate reflux, wherein the cross-sectional area is sufficiently small to restrict the flow of irrigation fluid to the aspiration manifold to allow enough irrigation fluid to continue to flow to the handpiece and into the patient's eye.

Yet in another embodiment, it is provided a surgical system for aspiration of a biological material which comprises a source of irrigation fluid, a collection cassette, a pump for creating a vacuum in the collection cassette, a handpiece applied to a surgical area for infusing irrigation fluid and for aspirating a biological material, an irrigation conduit connecting the handpiece to the source of irrigation fluid, a aspiration conduit connecting the handpiece to the collection cassette, a fluid vent conduit of which one end is connected to the irrigation conduit and the other end is connected to the aspiration conduit, and a safety device for maintaining a controlled fluid flow.

Further features and advantages of the present invention, as well as the structure and operation of various embodiments of the present invention, are described in detail below with reference to the accompanying drawings.

### BRIEF DESCRIPTION OF DRAWINGS

FIG. 1 is a diagrammatic view of one embodiment of a surgical system comprising a safety device in a fluid vent conduit for providing improved flow control.

FIG. 2 is a cross-sectional view of one embodiment of a fluid vent conduit comprising a safety device for providing improved flow control.

FIG. 3 is a perspective view of one embodiment of an ophthalmic surgical pump cassette for collecting aspirant fluid and tissue from a patient's eye containing a safety device for providing improved flow control.

FIG. 4 is a top view of an alternate embodiment of an ophthalmic surgical pump cassette of FIG. 3.

### DETAILED DESCRIPTION

The following description is merely exemplary in nature and is not intended to limit the present disclosure, application, or uses.

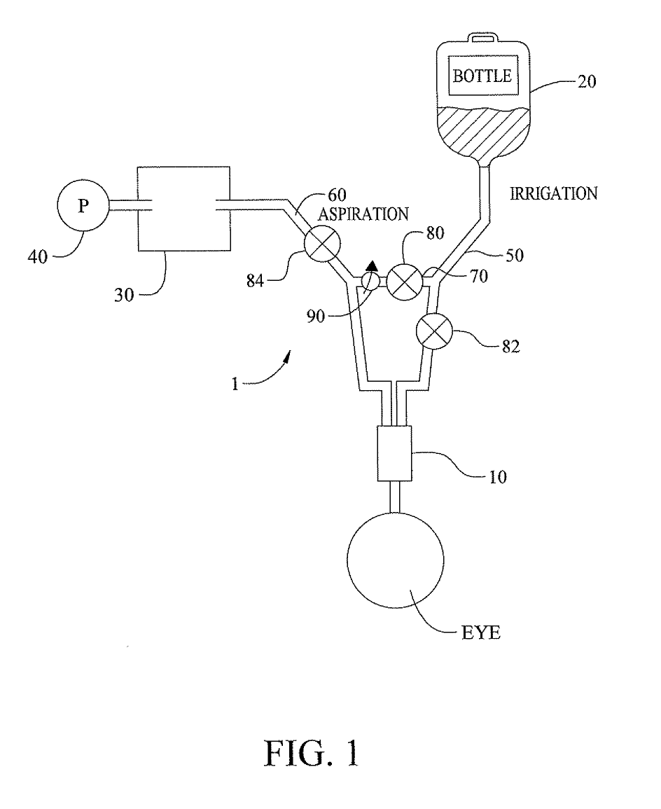

Referring to FIG. 1, an ophthalmic surgical system 1, in accordance with an embodiment, comprises a surgical handpiece 10, a source of irrigation fluid 20, a collection cassette 30, a vacuum pump 40, an irrigation conduit 50 connecting the surgical handpiece to the irrigation fluid source and an aspiration conduit 60 connecting the surgical handpiece to the vacuum pump 40 and the collection cassette 30, a fluid vent conduit 70 the ends of which ends are connected to the irrigation conduit 50 and the aspiration conduit 60 respectively, valves 80, 82, 84 disposed at various locations within the circuit, and a safety device 90 for maintaining a controlled fluid flow formed within the fluid vent conduit 70. The surgical system 1 is particularly useful in ophthalmic surgery where it is necessary to break up and remove undesirable biological materials from the patient's eye. In a particular embodiment, the surgical system 1 is used to remove cataract without causing irreparable damage to the eye.

A surgical handpiece 10 can be a conventional phacoemulsification handpiece comprising an operative tip, a withdrawal lumen and an annular sleeve for irrigation surrounding the withdrawal means. The surgical handpiece is placed on or into the surgical site to remove undesirable biological materials. In an ophthalmic surgical system, for example, the phaco handpiece 10 is inserted though an incision in an eye, and the operative tip coupled to an energy source applies energy, such as ultra-sound, to the surgical site to break up undesirable biological materials such as cataract. The surgical fluid is infused into the surgical site through the annular sleeve and the withdrawal means simultaneously aspirates fluids containing the undesirable materials away from the eye.

A source of irrigation fluid 20 typically includes a fluid container and surgical fluid. The surgical fluid can be any known surgical fluid and an ordinary skilled person in the art can select proper surgical fluid in accordance with the nature of the surgery to be operated. In an ophthalmic surgical system, the surgical fluid is ophthalmic surgical fluid such as, for example, BSS. Each end of the irrigation conduit 50 is connected to the container 20 and the phaco handpiece 10, respectively, so that the ophthalmic surgical fluid is delivered to the patient's eye through the irrigation sleeve of the phaco handpiece 10.

A collection cassette 30 typically has a collection chamber and an inlet and an outlet for connection to each of the handpiece 10 and the vacuum pump 40. The collection chamber accommodates biological debris aspirated from the surgical site via the withdrawal means of the handpiece 10 and the aspiration conduit 60. The collection cassette 30 can be selected from any collection means for a surgical system known in the art, regardless of its reusability. Thus, the cassette 30 can be any known reusable or disposable collection means. For safety and cleanliness of the operation, it may be preferable to select a collection cassette equipped with a fluid level detection device which is designed to prevent overflowing and leaking surgical fluids. The collection cassette 30 is installed in operative association with the handpiece 50 and the pump 40 by any means known in the art.

A vacuum pump 40 is connected to the collection cassette 30 and the handpiece 40 through the aspiration conduit 60 to provide the aspiration system comprising the handpiece, the conduit and the collection cassette with negative pressure or vacuum. The vacuum pump 40 can be any pump known in the art as long as it is suitable for a surgical system including the present surgical system. Preferably, the vacuum pump 40 is one suitable for an ophthalmic surgical system. Examples of a pump applicable to the present invention are, but not limited to, a rotary vane pump, a diaphragm pump, a scroll pump, a peristaltic pump and a vacuum or venturi pump.

A surgical system 1 typically requires two separate conduits 50 and 60 for the irrigation and aspiration system, respectively. An irrigation conduit 50 connects the surgical handpiece 10 to the irrigation fluid source 20 to provide the surgical site with the surgical fluid. The irrigation system may contain one or more valves placed between the handpiece 10 and the irrigation fluid source 20 to control the irrigation flow rate, thereby helping maintenance of a proper pressure of the surgical site.

An aspiration conduit 60 connects, for example, the surgical handpiece 10 to the collection cassette 30 and then to the vacuum pump 40, but it is obvious to an ordinary skilled person in the art that it is possible to modify the placement and the connection of the aspiration components. Both irrigation and aspiration conduits 50, 60 can be made of any known or conventional materials used for an ophthalmic surgical system. The vacuum pump 40 is operatively connected to the collection cassette 30 through the aspiration conduit 60, such that undesirable biological materials from the surgical site are aspirated to the collection cassette 30. The aspiration system may contain one or more valves placed between the handpiece 10 and the cassette/the pump to regulate the negative pressure, thereby helping maintenance of a proper pressure of the surgical site.

A fluid vent conduit 70 is designed to allow flow of an irrigation fluid into the aspiration conduit 60, thereby neutralizing negative pressure caused by a clogged or occluded aspiration tip. Typically, each end of the fluid vent conduit 70 is connected to the irrigation conduit 50 and the aspiration conduit 60 in a manner that an irrigation fluid can be supplied to the aspiration conduit 60. It would be obvious to an ordinary skilled person in the art that it is possible to modify the placement and the connection of the fluid vent conduit. A fluid vent conduit 70 can be made of any known tubing materials used for an ophthalmic surgical system. In an embodiment, a fluid vent conduit is made of silicone which may be advantageous for installing a pinch valve controlling flow rate inside the conduit.

Each of the irrigation conduit 50, the aspiration conduit 60, and the fluid vent conduit 70 can optionally have one or more valve controlling flow rate and/or air venting at various locations. The valves 80, 82, 84 shown in FIG. 1 are optional in nature and their locations and numbers may vary depending on the placement of a specific embodiment. In an embodiment, an ophthalmic surgical system has both at least one air vent valve and at least one fluid venting valve. Each valve can be controlled manually, electronically or electromechanically and may be connected to a controller for automatic operation.

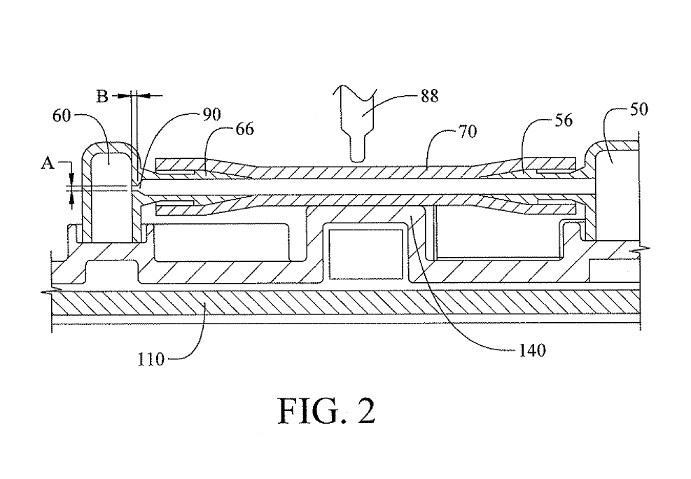

A safety device 90 for maintaining a controlled fluid flow is formed within the fluid vent conduit 70 or a manifold connecting the fluid vent conduit 70 and the aspiration conduit 60. The safety device 90 contains an orifice having an inlet for receiving a flow of fluid from the irrigation source 20 and an outlet for directing the flow of fluid into the aspiration conduit 60. The orifice needs to have a cross-sectional area sufficiently large to ensure an acceptable amount of fluid flows into the aspiration conduit. Such acceptable influx amount may have to be determined to satisfy various considerations of the present invention. For example, bypass flow through the fluid vent conduit 70 needs to reduce the vacuum level in the aspiration conduit 60 and the influx may have to provide adequate reflux. By utilizing infusion pressure applied to the infusion bottle 20, continuous reflux can be maintained. Continuous reflux is different than commonly known pulsed reflux created by a plunger valve pushing on an aspiration line or a reflux bulb. Also, the cross-section area needs to be sufficiently small to restrict the flow of fluid to allow enough fluid to continue to flow from the irrigation source 20 to the handpiece 10 at a surgical site. The safety device 90 is further illustrated below with reference to FIG. 2.

FIG. 2 shows a cross-section of one embodiment of a fluid vent system containing a safety device 90 for providing improved fluid control. A fluid vent conduit 70 is connected to an irrigation conduit or manifold 50 and an aspiration conduit or manifold 60, respectively, so that irrigation fluid flows from the irrigation conduit 50 to the aspiration conduit 60 through an orifice of the safety device 90. In an embodiment, a fluid vent conduit 70 is connected by manifolds forming a part of irrigation conduit 50 and an aspiration conduit 60 where each terminal portion of the fluid vent conduit 70 is inserted onto an arm 56 of an irrigation manifold and an arm 66 of an aspiration manifold, respectively.

The orifice of the safety device 90 can be molded into the aspiration manifold 66 of the ophthalmic surgical system. Generally, the orifice has a narrower interior than the fluid vent conduit 70 and is installed at an outlet for directing the flow of fluid into the aspiration conduit. As discussed previously, it is noted that the cross-sectional area of the orifice could be critical in maintaining a proper pressure inside the conduits of the surgical system. Therefore, the cross-sectional area needs to be sufficiently large to ensure an acceptable amount of fluid flows into the aspiration conduit and to be sufficiently small to restrict the flow of fluid to allow enough fluid to continue to flow from the irrigation source to a handpiece at a surgical site. In an embodiment, the cross-sectional area is between about 0.0002 and about 0.0004 square inches. In another embodiment, the orifice has a diameter (A of FIG. 2) of about 0.02 inches and a length (B of FIG. 2) of about 0.02 inches.

The safety device 90 may further contain a compressible length of tubing 70 connected to an aspiration manifold and an irrigation manifold. In an embodiment, the system optionally contains at least one valve 88, such as a pinch valve disposed along the fluid vent conduit 70. The length of tubing can be compressed by the pinch valve assembly for controlling the flow of fluid into the orifice. Where a pinch valve is installed, a block 140 can be formed beneath the fluid vent conduit 70 to provide contact surface for a pinch valve moving up and down to open and close the line.

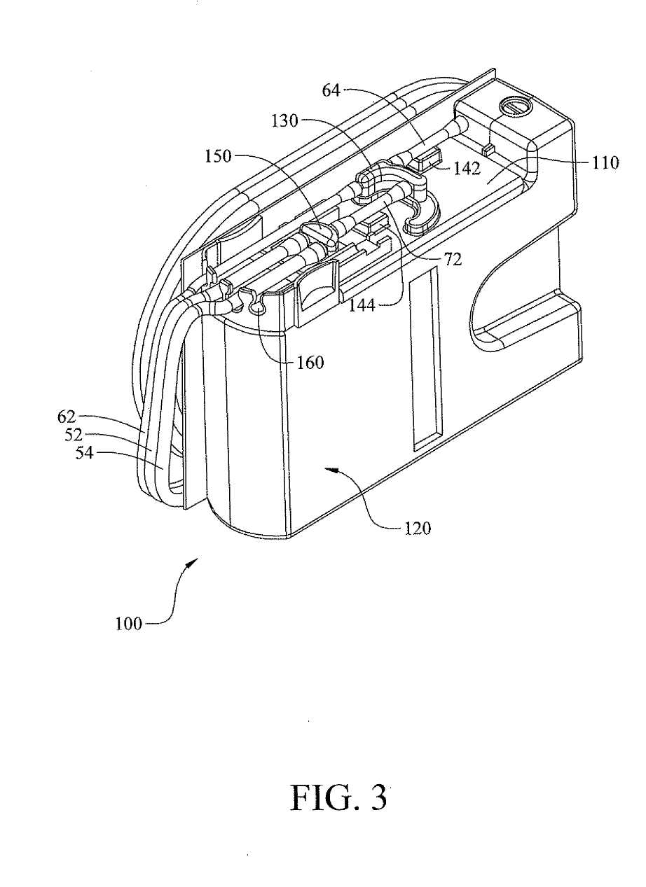

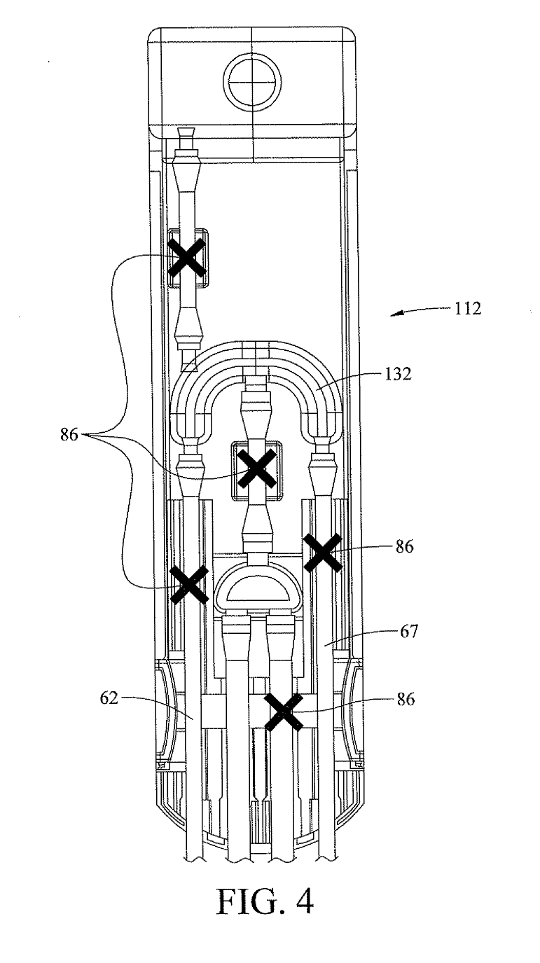

FIG. 3 shows a perspective view of one embodiment of a cassette in accordance with the present invention. An ophthalmic surgical pump cassette 100 for collecting aspirant fluid and tissue comprises an aspiration manifold 130, an irrigation manifold 150, an orifice (not shown in FIG. 3 but see FIG. 2 above) formed within the fluid vent conduit or the aspiration manifold, container 120 and manifold base 110. The cassette 100 is shown with an entire length of conduits 52, 54, and 62. The manifold base 110 and container 120 combine to form an ophthalmic surgical cassette, in accordance with the present invention. The manifold base 110 preferably includes an irrigation inlet conduit 54 for receiving irrigation flow from an irrigation source, such as is well known in the art. The manifold base 110 also preferably includes an irrigation outlet conduit 52 for allowing irrigation fluid to flow from the conduit 54 into an ophthalmic surgical instrument for use during surgery. The manifold base 110 further preferably includes aspiration conduits 62, 64 for delivering aspirant fluid and tissue from a surgical site to the container 120. The aspiration conduit 64 is connected to an inlet of the container. The manifold base 110 also preferably includes an aspiration manifold 130 connecting the aspiration conduits 62 and 64 with a liquid vent conduit 72. The aspiration manifold 130 allows valves of a surgical console (not shown) to control a flow of aspirant fluid and tissue into the cassette 100. Blocks 142 and 144 provide contact surfaces for conventional pinch valves to open and shut the liquid vent conduit 72 and the aspiration conduit 64 and are controlled via pump and surgical console in a conventional manner. The irrigation inlet conduit 54 also preferably includes and cooperates with a pinch valve for turning irrigation fluid flow on and off. The irrigation manifold 150 connects the liquid vent conduit 72 with the irrigation inlet conduit 54 and irrigation outlet conduit 52. The irrigation manifold 150 allows valves of the surgical console to control a flow of irrigation fluid from the source to the handpiece and between the irrigation manifold 150 and the aspiration manifold 130. The aspiration and irrigation manifolds 130, 150 may be removably attached to the cassette 100. FIG. 4 shows manifold base 110 with an additional empty slot 160 which can be used for an alternate embodiment of the present invention, described in detail below at FIG. 4. In an embodiment, an ophthalmic surgical pump cassette (not shown) contains an orifice molded into the aspiration manifold.

The manifold base 110 conveniently allows the irrigation inlet and outlet conduits 54 and 52 and the aspiration conduit 62 to remain connected to manifold base 110 as described above during an entire surgery. If the container 120 were to fill with fluid and tissue and need to be emptied, a single disconnection of the conduit 64 from the container 120 would allow an operator to empty container 120 and quickly reattach manifold base 110 to container 120 along with aspiration conduit 64. In this manner, container 120 can be quickly and conveniently emptied without the clumsy and often times difficult removal of conduits 52, 54, and 62 as would be required in the prior art.

FIG. 4 shows a top view of an alternate embodiment of a manifold base 112, in accordance with the present invention. The manifold base 112 is identical to the base 110 described above, with the exception that the manifold base 112 preferably includes a second aspiration conduit 86. The conduit 67 is connected to an alternate embodiment of a manifold 132, which is similar to the manifold 130 described above. Depending on the surgery performed, a surgeon may prefer a second aspiration conduit 67, as shown in FIG. 4 so that a second surgical handpiece can be attached without the need to remove the tubing from the surgical handpiece connected to an aspiration conduit 62. FIG. 4 also conveniently shows preferred optional pinch valve locations as indicated by the dark “X”s at 86.

When introducing elements or features and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

As various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments, but should be defined only in accordance with the following claims and their equivalents.

1. A safety device in a fluid vent conduit for reducing vacuum level in an aspiration conduit of an ophthalmic surgical system, the device comprising:
an orifice having an inlet for receiving a flow of fluid from an irrigation source;
wherein the orifice further has an outlet for directing the flow of fluid into the aspiration conduit;
wherein the orifice has a cross-sectional area sufficiently large to ensure an acceptable amount of fluid flows into the aspiration conduit, to reduce the vacuum level in the aspiration conduit, to an acceptable level in an acceptable amount of time and to provide adequate reflux; and
wherein the cross-section area is sufficiently small to restrict the flow of fluid to allow enough fluid to continue to flow from the irrigation source to a hand piece at a surgical site.

2. The safety device of claim 1, wherein the orifice is molded into an aspiration manifold of the ophthalmic surgical system.

3. The safety device of claim 2, further comprising a compressible length of tubing connected to the aspiration manifold and an irrigation manifold, wherein the length of tubing can be compressed by a pinch valve assembly for controlling the flow of fluid into the orifice.

4. The safety device of claim 2, wherein the cross-sectional area of the orifice is between about 0.0002 and about 0.0004 square inches.

5. The safety device of claim 1, wherein the orifice has a diameter of about 0.02 inches and a length of about 0.02 inches.

6. The safety device of claim 1, wherein the reflux is continuous.

7. An ophthalmic surgical pump cassette for collecting aspirant fluid and tissue from a patient's eye comprising:
an aspiration manifold formed on the cassette for connecting an aspiration conduit from a surgical handpiece to the cassette and for allowing valves of a surgical console to control a flow of aspirant fluid and tissue into the cassette;
an irrigation manifold formed on the cassette for connecting an irrigation conduit from a surgical handpiece to a source of irrigation fluid and for connecting a fluid vent conduit between the aspiration manifold and the irrigation manifold, the irrigation manifold also allowing valves of the surgical console to control a flow of irrigation fluid from the source to the handpiece and between the irrigation manifold and the aspiration manifold; and
an orifice formed within the fluid vent conduit or the aspiration manifold, the orifice having a cross-sectional area sufficiently large to ensure an acceptable amount of fluid flows into the aspiration manifold to reduce a vacuum level in the aspiration conduit, to an acceptable level in an acceptable amount of time, and to provide adequate reflux, wherein the cross-sectional area is sufficiently small to restrict the flow of irrigation fluid to the aspiration manifold to allow enough irrigation fluid to continue to flow to the handpiece and into the patient's eye.

8. The ophthalmic surgical pump cassette of claim 7, wherein the orifice is molded into the aspiration manifold.

9. The ophthalmic surgical pump cassette of claim 7, wherein the cross-sectional area is between about 0.0002 and about 0.0004 square inches.

10. The ophthalmic surgical pump cassette of claim 7, wherein the orifice has a diameter of about 0.02 inches and a length of about 0.02 inches.

11. The ophthalmic surgical pump cassette of claim 7, wherein the aspiration and irrigation manifolds are removably attached to the cassette.

12. The ophthalmic surgical pump cassette of claim 7, wherein the reflux is continuous.

13. A surgical system for aspiration of a biological material comprising:
a handpiece applied to a surgical area for infusing irrigation fluid and for aspirating a biological material;
a source of irrigation fluid;
a collection cassette;
a pump for creating a vacuum in the collection cassette;
an irrigation conduit connecting the handpiece to the source of irrigation fluid;
an aspiration conduit connecting the handpiece to the collection cassette;
a fluid vent conduit of which one end is connected to the irrigation conduit and the other end is connected to the aspiration conduit; and
a safety device of formed within the fluid vent conduit or a junction of the fluid vent conduit and the aspiration conduit to provide improved fluid control.

14. The surgical system of claim 13, wherein a valve is formed within at least one of the irrigation conduit, the aspiration conduit, and the fluid vent conduit.

15. The surgical system of claim 13, wherein the valve is a pinch valve.

16. The surgical system of claim 13, wherein the safety device comprises:
an orifice having an inlet for receiving a flow of fluid from an irrigation source;
wherein the orifice further has an outlet for directing the flow of fluid into the aspiration conduit;
wherein the orifice has a cross-sectional area sufficiently large to ensure an acceptable amount of fluid flows into the aspiration conduit, to reduce the vacuum level in the aspiration conduit, to an acceptable level in an acceptable amount of time and to provide adequate reflux; and
wherein the cross-section area is sufficiently small to restrict the flow of fluid to allow enough fluid to continue to flow from the irrigation source to a handpiece at a surgical site.
